(19) United States
(12) Patent Application Publication
(10) Pub. No.: US 2011/0177584 A1
(43) Pub. Date: Jul. 21, 2011

(54) ACOUSTIC WAVE SENSOR AND DETECTION METHOD USING ACOUSTIC WAVE SENSOR

(75) Inventors: Hiroyasu KADOYA, Nagaokakyo-shi (JP); Koji FUJIMOTO, Nagaokakyo-shi (JP); Kansho YAMAMOTO, Nagaokakyo-shi (JP)

(73) Assignee: MURATA MANUFACTURING CO., LTD., Nagaokakyo-shi (JP)

(21) Appl. No.: 13/076,519

(22) Filed: Mar. 31, 2011

Related U.S. Application Data
(63) Continuation of application No. PCT/JP2009/005076, filed on Oct. 1, 2009.

(30) Foreign Application Priority Data
Oct. 7, 2008 (JP) 2008-260996

(51) Int. Cl. G01N 29/12; C12M 1/34

(52) U.S. Cl. 435/287.1; 422/68.1

(57) ABSTRACT

An acoustic wave sensor having significantly increased sensitivity and excellent sensing reproducibility and stability includes a piezoelectric substrate, an acoustic wave element including an electrode disposed on the piezoelectric substrate, and a reactive membrane which overlies the acoustic wave element and which is reduced in mass by direct or indirect chemical reaction with a measured substance. The measured substance is detected such that a change in mass applied to the acoustic wave element from the reactive membrane is detected by a change in frequency.

### BACKGROUND OF THE INVENTION

1. Field of the Invention

The present invention relates to acoustic wave sensors and detection methods using the acoustic wave sensors. The present invention particularly relates to an acoustic wave sensor based on a change in frequency of an acoustic wave element and a detection method using such an acoustic wave sensor.

2. Description of the Related Art

Previously, acoustic wave sensors based on the fact that the frequency response of acoustic wave elements is varied by the reaction of reactive substances, such as antibodies, arranged on surfaces of the acoustic wave elements have been developed. The acoustic wave sensors perform sensing such that the frequencies of the acoustic wave elements are varied by the reaction of ligands or receptors, such as antibodies, solid-phased on the acoustic wave elements with antigens contained in samples.

For example, an acoustic wave sensor shown in FIGS. 12A and 12B, which are schematic views, contains an oligonucleotide (—ON′) immobilized on an electrode 12 disposed on a sub-family of a crystal oscillator 11 which is an acoustic wave element. A reagent contains a complementary base sequence (—ON) specifically bound to the oligonucleotide immobilized on the electrode 12 and a Fab′ domain 16 or IgG domain 18 bound to an antigen 14. The antigen 14 is bound to the crystal oscillator with the reagent (see, for example, Japanese Unexamined Patent Application Publication No. 9-292397).

For a configuration in which a ligand or a receptor such as an antibody is solid-phased on an acoustic wave element, the sensitivity of an acoustic wave sensor depends on the amount of the ligand or receptor solid-phased on the acoustic wave element. Since there is a limitation on increases in the amount of the ligand or receptor solid-phased on the acoustic wave element, it is difficult to increase the sensitivity of the acoustic wave sensor.

Sensing reproducibility or stability may be possibly reduced by the inhibition of reaction by steric hindrance or by the capture of a substance in a liquid containing a sample.

### SUMMARY OF THE INVENTION

In view of the foregoing, preferred embodiments of the present invention provide an acoustic wave sensor which has a significantly increased sensitivity and which has good sensing reproducibility and stability, and also provide a detection method using such an acoustic wave sensor.

An acoustic wave sensor according to a preferred embodiment of the present invention includes a piezoelectric substrate, an acoustic wave element including electrodes disposed on the piezoelectric substrate, and a reactive membrane which overlies the acoustic wave element and which is reduced in mass by direct or indirect chemical reaction with a measured substance. The measured substance is detected such that the change in mass applied to the acoustic wave element from the reactive membrane is detected by a change in frequency.

In the above configuration, the reduction in mass of the reactive membrane caused by the direct or indirect chemical reaction with the measured substance varies the mass applied to the acoustic wave element from the reactive membrane to vary the frequency of the acoustic wave element. Thus, the measured substance can be detected.

In the case of detecting the measured substance from the increase in mass of the reactive membrane, the increase of sensitivity is difficult because of limitations on the solid-phasing of ligands or receptors such as antibodies. However, in the configuration of this preferred embodiment of the present invention, the measured substance can be detected until the reactive membrane is lost and a larger change in frequency can be achieved. This allows the acoustic wave sensor to have increased sensitivity. The acoustic wave sensor does not utilize steric hindrance or the like and therefore has good sensing reproducibility and stability.

It is preferred that the measured substance be combined with an enzyme by an immunological method and the mass of the reactive membrane be reduced by the direct or indirect chemical reaction with the enzyme.

In this case, the reduction in mass of the reactive membrane by the direct or indirect chemical reaction with the enzyme combined with measured substance varies the mass applied to the acoustic wave element from the reactive membrane to vary the frequency of the acoustic wave element; hence, the measured substance can be detected.

Since the enzyme functions as a catalyst, the chemical reaction of the reactive membrane continues until the reactive membrane is lost. As a result, a larger change in frequency is achieved as compared to the case where an antibody is placed on a conventional acoustic wave element. This allows the acoustic wave sensor to have high sensitivity.

The amount of an antigen contained in a sample can be accurately detected such that the mass of the reactive membrane of the acoustic wave sensor is reduced by the chemical reaction with an enzyme of an enzyme-labeled antibody that has captured the antigen in the sample. The type of the antigen in the sample can be accurately determined such that the enzyme-labeled antibody is selected depending on the antigen in the sample. Therefore, sensing stability and reproducibility are excellent.

The electrodes are preferably IDT electrodes, for example. This allows the sensitivity of detection to be increased by an increase in frequency.

The acoustic wave element is preferably a surface acoustic wave element, for example. This allows the acoustic wave sensor to have a smaller size and higher sensitivity.

The reactive membrane is preferably an organic membrane, for example. This allows the reactive membrane to be formed at low cost.

The reactive membrane is preferably made of a biodegradable plastic, for example. This is suitable for biological measured substances.

The reactive membrane is preferably an inorganic membrane, for example. This allows the reactive membrane to be stably formed and therefore allows a variation in the acoustic wave sensor to be reduced.

The reactive membrane is preferably a ZnO membrane, for example. This allows the reactive membrane to be formed at low cost.

In order to solve the above problems, preferred embodiments of the present invention provide a detection method using an acoustic wave sensor configured as described below.

The detection method using the acoustic wave sensor according to a preferred embodiment of the present invention is a detection method using an acoustic wave sensor having any one of the above configurations. The detection method using the acoustic wave sensor includes (i) a step of mixing a sample, an enzyme-labeled antibody containing an enzyme and an antibody capturing an antigen contained in the sample, and a medium-labeled antibody containing a medium and the antibody capturing the antigen in the sample such that the enzyme-labeled antibody and the medium-labeled antibody are combined together with the antigen sandwiched therebetween; (ii) a step of providing the enzyme-labeled antibody combined with the antigen and the medium-labeled antibody on the reactive membrane of the acoustic wave sensor; and (iii) a step of detecting the change in frequency of the acoustic wave sensor such that the mass of the reactive membrane of the acoustic wave sensor is reduced by the direct or indirect chemical reaction with the enzyme of the enzyme-labeled antibody.

According to the above method, the mass of the reactive membrane of the acoustic wave sensor can be reduced by the chemical reaction with the enzyme-labeled antibody that has captured the antigen in the sample and therefore the amount of the antigen in the sample can be accurately detected.

The type of the antigen in the sample can be accurately determined such that the enzyme-labeled antibody is selected depending on the antigen in the sample.

In order to solve the above problems, various preferred embodiments of the present invention provide a detection method using another acoustic wave sensor configured as described below.

The detection method using the acoustic wave sensor is a detection method using an acoustic wave sensor having any one of the above configurations. The detection method using the acoustic wave sensor includes (i) a step of mixing a sample and an enzyme-labeled antibody containing an enzyme and an antibody capturing an antigen contained in the sample such that the antibody of the enzyme-labeled antibody captures the antigen, (ii) a step of combining the enzyme-labeled antibody that has captured the antigen and an antibody attached to a medium placed near the reactive membrane together with the antigen sandwiched therebetween, (iii) a step of producing a reactant reacting with the reactive membrane by the action of the enzyme of the enzyme-labeled antibody such that the reactant is provided near the acoustic wave sensor, and (iv) a step of detecting the change in frequency of the acoustic wave sensor such that the mass of the reactive membrane of the acoustic wave sensor is reduced by the direct or indirect chemical reaction with the reactant.

According to this method, the mass of the reactive membrane of the acoustic wave sensor can be reduced by the chemical reaction with the reactant produced by the action of the enzyme of the enzyme-labeled antibody that has captured the antigen in the sample and therefore the amount of the antigen in the sample can be accurately detected.

The type of the antigen in the sample can be accurately determined such that the enzyme-labeled antibody is selected depending on the antigen in the sample.

An acoustic wave sensor according to a preferred embodiment of the present invention is capable of being increased in sensitivity and is excellent in sensing reproducibility and stability.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1 is a sectional view of a principal portion of an acoustic wave sensor according to a first preferred embodiment of the present invention.

FIG. 2 is a sectional view of a principal portion of an acoustic wave element according to the first preferred embodiment of the present invention.

FIGS. 3A-3C are schematic views showing a detection method using an acoustic wave sensor according to a second preferred embodiment of the present invention.

FIGS. 4A-4C are schematic views showing a detection method using an acoustic wave sensor according to the second preferred embodiment of the present invention.

FIGS. 5A-1 to 5B-2 are schematic views showing a detection method using an acoustic wave sensor according to a third preferred embodiment of the present invention.

FIG. 6 is a sectional view of a principal portion of an acoustic wave sensor according to a fourth preferred embodiment of the present invention.

FIG. 7 is a schematic view showing a detection method using an acoustic wave sensor according to a fifth preferred embodiment of the present invention.

FIG. 8 is a graph showing the change in oscillation frequency according to the fifth preferred embodiment of the present invention.

FIG. 9 is a graph showing the relationship between the maximum rate of change in oscillation frequency and the concentration of an enzyme according to the fifth preferred embodiment of the present invention.

FIG. 10 is a graph showing the frequency response before and after reaction according to the fifth preferred embodiment of the present invention.

FIG. 11 is a sectional view of a principal portion of an acoustic wave sensor according to a six preferred embodiment of the present invention.

FIGS. 12A and 12B are schematic views showing a conventional detection method using an acoustic wave sensor.

### DETAILED DESCRIPTION OF THE PREFERRED EMBODIMENTS

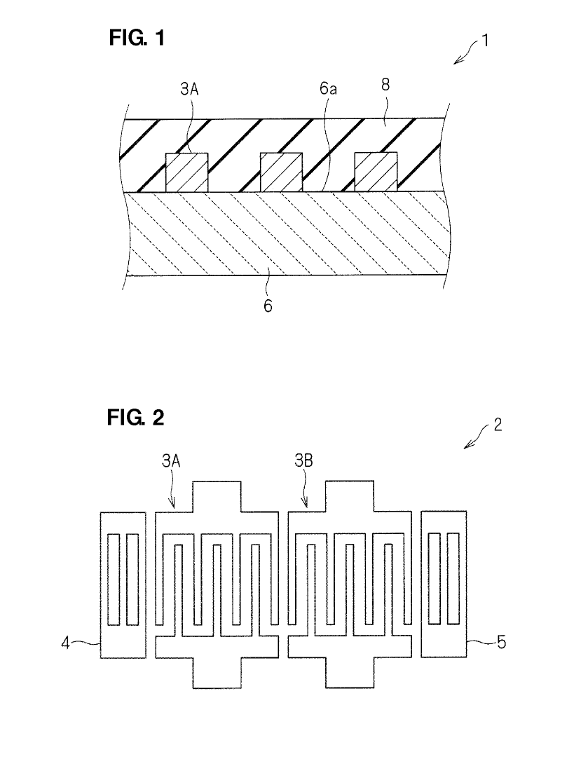

Preferred embodiments of the present invention will now be described with reference to FIGS. 1 to 11.

### First Preferred Embodiment

The configuration of an acoustic wave sensor 1 of a first preferred embodiment of the present invention is described below with reference to FIGS. 1 and 2. FIG. 1 is a sectional view of a principal portion of the acoustic wave sensor 1. FIG. 2 is a sectional view of a principal portion of an acoustic wave element 2.

With reference to FIG. 1, the acoustic wave sensor 1 is configured such that the acoustic wave element 2, which is shown in FIG. 2, is disposed on a principal surface 6a of a piezoelectric substrate 6 and a reactive membrane 8 extends over the acoustic wave element 2.

The piezoelectric substrate 6 is preferably made of single-crystalline dielectric such as LiTaO₃, LiNaO₃, or quartz, for example.

With reference to FIG. 2, the acoustic wave element 2 includes comb-shaped IDT (interdigital transducer) electrodes 3A and 3B arranged to excite a surface acoustic wave and also includes reflectors 4 and 5 that are arranged on both sides of a region containing the IDT electrodes 3A and 3B in the propagation direction of the surface acoustic wave. The IDT electrodes 3A and 3B and the reflectors 4 and 5 are preferably made of Al, Au, Pt, Cu, Ag, or an alloy containing these metals, for example.

The acoustic wave element 2 is not limited to one using a surface acoustic wave and may be an element using a bulk acoustic wave. When the acoustic wave element 2 is one using a surface acoustic wave, the acoustic wave sensor may have a smaller size and higher sensitivity.

Electrodes for use in the acoustic wave element 2 are preferably IDT electrodes, which can be increased in detection sensitivity by the increase of frequency, and may be different in shape from such IDT electrodes.

The reactive membrane 8 extends over the IDT electrodes 3A and 3B and reflectors 4 and 5 of the acoustic wave element 2. The reactive membrane 8 is preferably made of a material selected depending on an enzyme such that the mass thereof is varied by the direct or indirect chemical reaction with the enzyme combined with an antigen. When the enzyme is, for example, a protease, the reactive membrane 8 is preferably a protein membrane. When the enzyme is an aldehyde-degrading enzyme, the reactive membrane 8 is preferably a ZnO membrane.

In the case of forming the reactive membrane 8 as an inorganic membrane, the reactive membrane 8 can be stably formed. This allows a variation in the acoustic wave sensor 1 to be reduced. In particular, the ZnO membrane can be formed at low cost. In the case of forming the reactive membrane 8 in the form of an organic membrane, the reactive membrane 8 can be formed at low cost.

The reactive membrane 8 chemically reacts with the enzyme. The chemical reaction varies the mass of the reactive membrane 8 and also varies the frequency of the acoustic wave sensor 1. The change in frequency of the acoustic wave sensor 1 is measured. The presence of an antigen combined with the enzyme is detected by the change in frequency thereof. That is, a mass is applied to a vibration-propagating region containing the IDT electrodes 3A and 3B and the reflectors 4 and 5 from the reactive membrane 8. The change in mass of the reactive membrane 8 caused by the chemical reaction varies vibration properties of a surface acoustic wave propagating on the vibration-propagating region. Changes in vibration properties thereof are input to one of the IDT electrodes 3A and 3B and are output from the other, whereby the gain of each frequency is measured. This allows the change in mass of the reactive membrane 8 caused by the chemical reaction, that is, the presence or amount of an antigen, to be detected.

The acoustic wave sensor 1 is unlike conventional sensors in that no antibody is placed on the acoustic wave element 2. Therefore, the chemical reaction of the reactive membrane 8 with the enzyme continues until the reactive membrane 8 is lost. As a result, a larger change in frequency is achieved. This allows the acoustic wave sensor 1 to have high sensitivity.

Since the reactive membrane 8 can be formed in advance so as to serve as a load to the acoustic wave element 2, an applied mass is uniform and a difference in shape can be reduced. Therefore, variations in sensing can be reduced.

### Second Preferred Embodiment

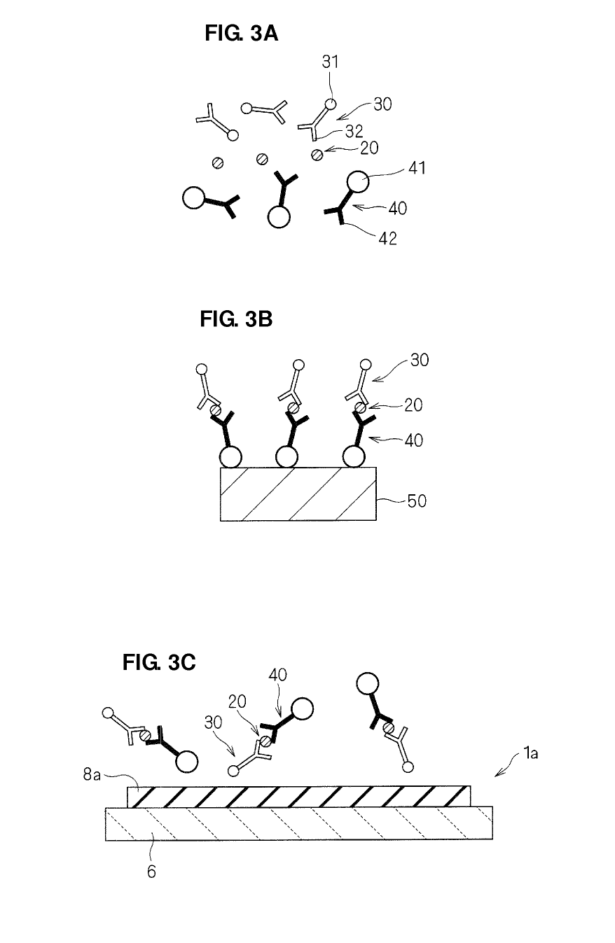

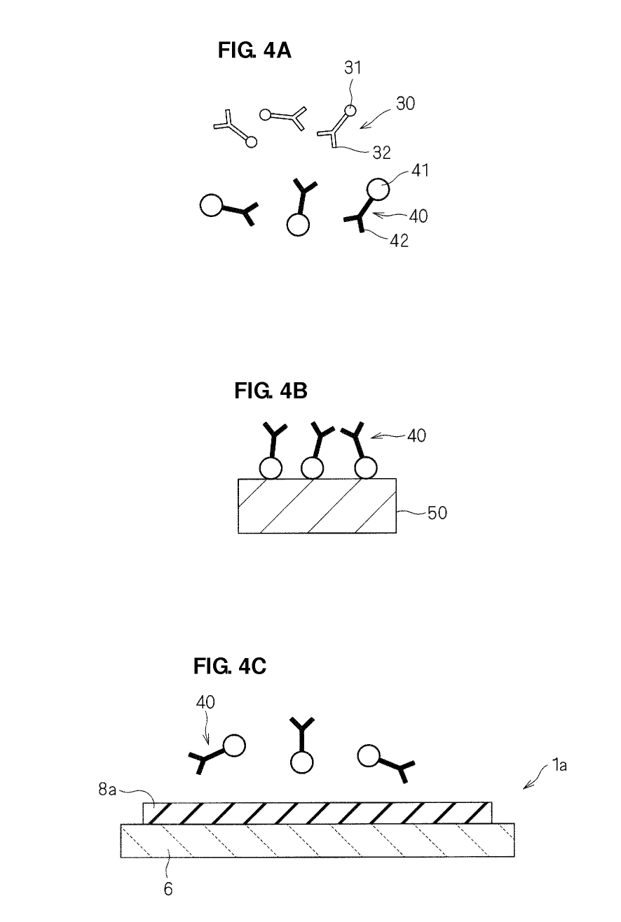

A detection method using an acoustic wave sensor having the configuration described in the first preferred embodiment will now be described with reference to FIGS. 3A-3C and 4A-4C.

FIGS. 3A-3C and 4A-4C are schematic views illustrating the detection method. FIGS. 3A-3c show the case where an antigen 20 is present. FIGS. 4A-4C show the case where no antigen 20 is present.

As shown in FIGS. 3C and 4C, an acoustic wave sensor 1a used in the second preferred embodiment of the present invention includes a protein membrane 8a which overlies a piezoelectric substrate 6 and an acoustic wave element (not shown) and which is made of a protein.

The detection method of the second preferred embodiment of the present invention includes the steps described below.

As shown in FIGS. 3A and 4A, a sample that may possibly contain the antigen 20, a protease-labeled antibody 30 that contains a protease 31 which is an enzyme chemically reacting with a protein and also contains an antibody 32 capturing the antigen 20, and a magnetic bead-labeled antibody 40 containing a magnetic bead 41 corresponding to a medium and an antibody 42 capturing the antigen 20 are mixed together, whereby the protease-labeled antibody 30 and the magnetic bead-labeled antibody 40 are combined together with the antigen 20 sandwiched therebetween.

As shown in FIGS. 3B and 4B, the magnetic bead-labeled antibody 40 is aggregated with a magnet 50. The magnetic bead-labeled antibody 40 is washed while being held with the magnet 50.

When the antigen 20 is present in the sample, the protease-labeled antibody 30 and the magnetic bead-labeled antibody 40 are combined together with the antigen 20 sandwiched therebetween as shown in FIG. 3B. As a result, the protease-labeled antibody 30, the antigen 20, and the magnetic bead-labeled antibody 40 are attracted by the magnet 50 and are held with the magnet 50 in such a state that the protease-labeled antibody 30, the antigen 20, and the magnetic bead-labeled antibody 40 are combined together. In contrast, when no antigen is present in the sample, the protease-labeled antibody 30 cannot be combined with the magnetic bead-labeled antibody 40 as shown in FIG. 4B. As a result, the magnetic bead-labeled antibody 40 only is attracted by the magnet 50 and is held with the magnet 50.

The magnet 50 is moved onto the protein membrane 8a of the acoustic wave sensor 1a and the frequency of the acoustic wave sensor 1a is measured.

When the antigen 20 is present in this step, the protease 31 in the protease-labeled antibody 30 containing the antibody 32 that has captured the antigen 20 chemically reacts with the protein membrane 8a of the acoustic wave sensor 1a as shown in FIG. 3C. This varies the frequency of the acoustic wave sensor 1a.

In contrast, when the antigen 20 is not present, the protease-labeled antibody 30 containing the enzyme 31 chemically reacting with the protein membrane 8a is not present as shown in FIG. 4C and therefore the frequency of the acoustic wave sensor 1a is not varied.

Thus, the amount of the antigen 20 can be determined by measuring the change in frequency of the acoustic wave sensor 1a.

According to the detection method, the protein membrane 8a, which is made of the protein, is removed by the reaction with the protease 31, which is an enzyme, and therefore the frequency is varied. Therefore, the change in load applied to the acoustic wave sensor 1a depends on the amount of the protein in the protein membrane 8a formed in advance. As a result, high sensitivity is achieved.

Since the magnetic bead-labeled antibody 40 is combined only with the protease-labeled antibody 30 that has captured the antigen 20, only the protease-labeled antibody 30 that has captured the antigen 20 can be moved onto the protein membrane 8a of the acoustic wave sensor 1a with the magnetic bead-labeled antibody 40. That is, the protease-labeled antibody 30 that has not captured the antigen 20 is not moved onto the protein membrane 8a of the acoustic wave sensor 1a. Therefore, any reaction is not inhibited by steric hindrance or a substance in a liquid is not captured. As a result, sensing stability and reproducibility are excellent.

### Third Preferred Embodiment

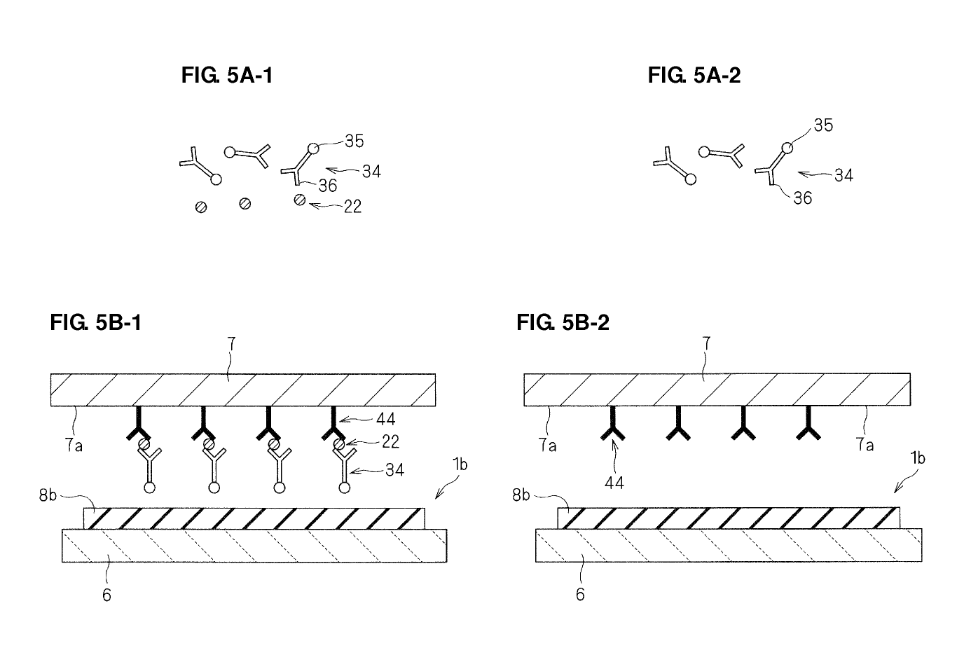

A detection method using the acoustic wave sensor according to a third preferred embodiment of the present invention will now be described with reference to schematic views in FIGS. 5A-1 to 5B-2. FIGS. 5A-1 and 5B-1 show the case where an antigen 22 is present. FIGS. 5A-2 and 5B-2 show the case where no antigen 22 is present.

As shown in FIGS. 5B-1 and 5B-2, an acoustic wave sensor 1b used in the third preferred embodiment of the present invention is preferably configured such that a ZnO membrane 8b overlies a piezoelectric substrate 6 and an acoustic wave element (not shown) and corresponds to a reactive membrane.

The acoustic wave sensor 1b is configured such that the following antibody is placed on an inner surface 7a of a lid 7 disposed around the ZnO membrane 8b, which is a medium, and/or an inner surface (not shown) of a side wall of the lid 7: an antibody 44 that immobilizes the antigen 22 in a sample in such a state that the antigen 22 is captured by an antibody 36 contained in an aldehyde-degrading enzyme-labeled antibody 34.

The detection method of the third preferred embodiment of the present invention includes steps below.

As shown in FIGS. 5A-1 and 5A-2, a sample that may possibly contain the antigen 22 is mixed with the aldehyde-degrading enzyme-labeled antibody 34 that contains an aldehyde-degrading enzyme 35 which is an enzyme producing an acid corresponding to a reactant chemically reacting with the ZnO membrane 8b and that contains the antibody 36 capturing the antigen 22. This combines the antigen 22 with the aldehyde-degrading enzyme-labeled antibody 34 when the antigen 22 is contained in the sample.

As shown in FIGS. 5B-1 and 5B-2, a mixture of the sample and the aldehyde-degrading enzyme-labeled antibody 34 is provided near the reactive membrane 8 of the acoustic wave sensor 1b, whereby the antibody 44 solid-phased on the inside of the lid 7 of the acoustic wave sensor 1b and/or the inside of the side wall of the lid 7 is combined only with the aldehyde-degrading enzyme-labeled antibody 34 that has captured the antigen 22. The change in frequency of the acoustic wave sensor 1b is then detected.

When the antigen 22 is present in the sample, the protease-labeled antibody 34 is immobilized to the antibody 44 with the antigen 22 as shown in FIG. 5B-1. The acid, which corresponds to a reactant, is produced by the reaction of the aldehyde-degrading enzyme 35, which is contained in the aldehyde-degrading enzyme-labeled antibody 34 immobilized with the antigen 22, with aldehyde provided near the ZnO membrane 8b of the acoustic wave sensor 1b. The acid chemically reacts with the ZnO membrane 8b of the acoustic wave sensor 1b. This varies the frequency of the acoustic wave sensor 1b.

In contrast, when no antigen 20 is present in the sample, the aldehyde-degrading enzyme-labeled antibody 34 is not immobilized to the antibody 44 and therefore flows off. As a result, the frequency of the acoustic wave sensor 1b is not varied.

Thus, the amount of the antigen 22 can be determined by measuring the change in frequency of the acoustic wave sensor 1b.

According to the detection method, the acid is produced by the action of the aldehyde-degrading enzyme-labeled antibody 34 and the ZnO membrane 8b is removed by the acid, whereby the frequency is varied. Therefore, the change in load applied to the acoustic wave sensor 1b depends on the amount of the ZnO membrane 8b formed in advance. As a result, high sensitivity is achieved.

Since the antibody 44 is combined only with the aldehyde-degrading enzyme-labeled antibody 34 that has captured the antigen 22, only the aldehyde-degrading enzyme-labeled antibody 34 that has captured the antigen 22 can be held near the ZnO membrane 8b of the acoustic wave sensor 1b with the antibody 44. That is, the aldehyde-degrading enzyme-labeled antibody 32 that has not captured the antigen 22 is not held near the ZnO membrane 8b of the acoustic wave sensor 1b. Therefore, any reaction is not inhibited by steric hindrance or a substance in a liquid is not captured. As a result, sensing stability and reproducibility are excellent.

### Fourth Preferred Embodiment

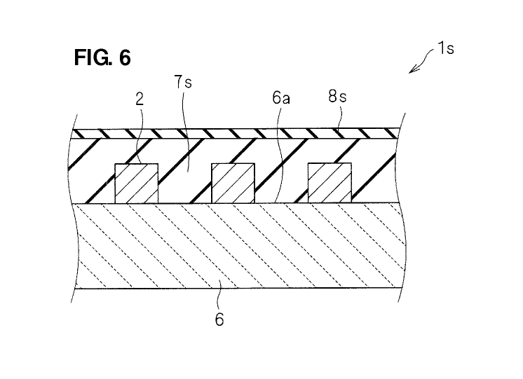

The configuration of an acoustic wave sensor 1s of a fourth preferred embodiment of the present invention will now be described with reference to FIG. 6.

The acoustic wave sensor 1s of the fourth preferred embodiment of the present invention is substantially identical in configuration to the acoustic wave sensor 1 of the first preferred embodiment of the present invention. The same members or portions as those described in the first preferred embodiment of the present invention are denoted by the same reference numerals as those used in the first preferred embodiment of the present invention. Differences therebetween are mainly described below.

FIG. 6 is a sectional view of a principal portion of the acoustic wave sensor 1s. With reference to FIG. 6, the acoustic wave sensor 1s of Example 4, as well as the acoustic wave sensor 1 of the first preferred embodiment of the present invention, is configured such that an acoustic wave element 2 is disposed on a principal surface 6a of a piezoelectric substrate 6.

The acoustic wave sensor 1s of the fourth preferred embodiment of the present invention is different from the acoustic wave sensor 1 of the first preferred embodiment of the present invention in that an insulating film 7s is disposed on the principal surface 6a of the piezoelectric substrate 6 so as to cover the acoustic wave element 2 and a reactive membrane 8s is disposed on the insulating film 7s.

The acoustic wave sensor 1s of the fourth preferred embodiment of the present invention, as well as the acoustic wave sensor 1 of the first preferred embodiment of the present invention, measures the change in frequency due to the change in mass of the reactive membrane 8s by a chemical reaction.

The acoustic wave sensor 1s of the fourth preferred embodiment of the present invention is unlike conventional sensors in that no antibody is placed on the acoustic wave element 2. Therefore, the chemical reaction of the reactive membrane 8s with an enzyme continues until the reactive membrane 8s is lost. As a result, a larger change in frequency is achieved. This allows the acoustic wave sensor 1s to have high sensitivity.

Since the reactive membrane 8s can be formed in advance so as to serve as a load to the acoustic wave element 2, an applied mass is uniform and a difference in shape can be reduced. Therefore, variations in sensing can be prevented and minimized.

Since the acoustic wave element 2 is protected with the insulating film 7s after the reactive membrane 8s is lost, measurement can be stably performed to the end. The acoustic wave sensor 1s can be repeatedly used by forming the reactive membrane 8s on the insulating film 7s again.

### Fifth Preferred Embodiment

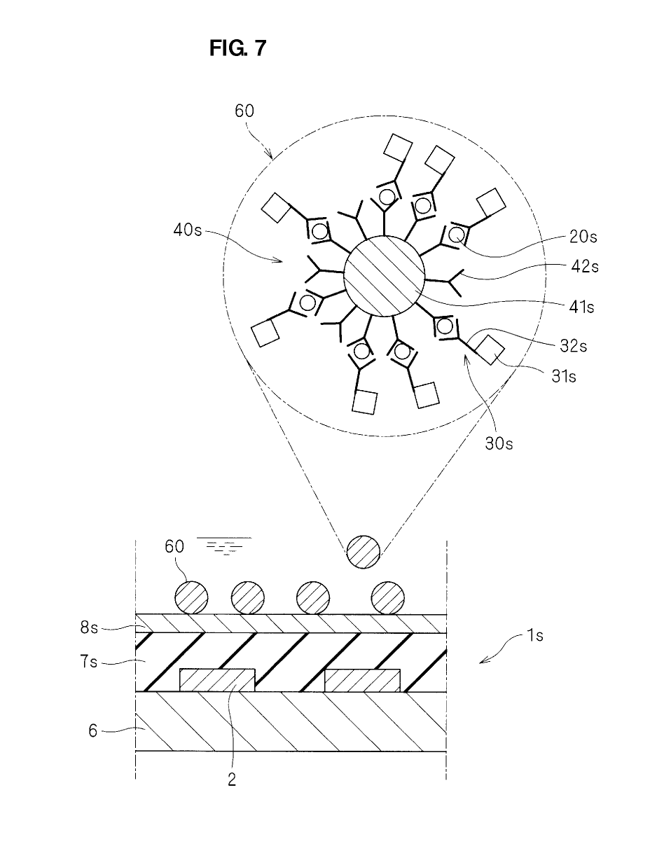

A detection method according to a fifth preferred embodiment of the present invention using the acoustic wave sensor 1s of the fourth preferred embodiment of the present invention will now be described with reference to FIGS. 7 to 10.

FIG. 7 is a schematic view illustrating the detection method. The detection method of Example 5 is used to measure the frequency of the acoustic wave sensor 1s such that a complex 60 containing an enzyme 31s reacting with the reactive membrane 8s is moved onto the reactive membrane 8s of the acoustic wave sensor 1a.

The detection method of the fifth preferred embodiment of the present invention includes steps below.

First, the following substances are mixed together: (a) a sample that may possibly contain an antigen 20s, (b) an enzyme-labeled antibody 30s containing the enzyme 31s reacting with the reactive membrane 8s and an antibody 32s which captures the antigen 20s and which is immobilized to the enzyme 31s, and (c) a magnetic bead-labeled antibody 40s containing a magnetic bead 41s and an antibody 42s which captures the antigen 20s and which is immobilized to the magnetic bead 41s. This combines the enzyme-labeled antibody 30s and the magnetic bead-labeled antibody 40s together with the antigen 20s sandwiched therebetween to form the complex 60.

Next, the magnetic bead-labeled antibody 40s, including the magnetic bead-labeled antibody 40s contained in the complex 60, is aggregated with a magnet and is then washed while being held with magnet.

When the antigen 20s is present in the sample, the enzyme-labeled antibody 30s and the magnetic bead-labeled antibody 40 are combined together with the antigen 20s sandwiched therebetween. As a result, the complex 60 is attracted by the magnet and is held with the magnet in such a state that the enzyme-labeled antibody 30s, the antigen 20s, and the magnetic bead-labeled antibody 40s are combined together. In contrast, when no antigen 20s is present in the sample, the enzyme-labeled antibody 30s cannot be combined with the magnetic bead-labeled antibody 40s. As a result, the magnetic bead-labeled antibody 40s only is attracted by the magnet and is held with the magnet.

Next, the magnetic bead-labeled antibody 40s, including the magnetic bead-labeled antibody 40s contained in the complex 60, is moved onto the reactive membrane 8s of the acoustic wave sensor is. The frequency of the acoustic wave sensor is then measured.

When the antigen 20s is present in the sample, the enzyme 31s in the enzyme-labeled antibody 30s containing the antibody 32s that has captured the antigen 20s reacts with the reactive membrane 8s of the acoustic wave sensor 1s and therefore the frequency of the acoustic wave sensor is varied.

In contrast, when no antigen 20s is present in the sample, the enzyme-labeled antibody 30s containing the enzyme 31s reacting with the reactive membrane 8s is not present and therefore the frequency of the acoustic wave sensor is not varied.

Thus, the presence or amount of the antigen 20s can be determined by detecting the change in frequency of the acoustic wave sensor is.

According to the detection method, the reactive membrane 8s is removed by the reaction with the enzyme 31s, whereby the frequency is varied. Therefore, the change in load applied to the acoustic wave sensor is depends on the amount of the reactive membrane 8s formed in advance. As a result, high sensitivity is achieved.

Since the magnetic bead-labeled antibody 40s is combined only with the enzyme-labeled antibody 30s that has captured the antigen 20s, only the enzyme-labeled antibody 30s that has captured the antigen 20s can be moved onto the reactive membrane 8s of the acoustic wave sensor is with the magnetic bead-labeled antibody 40s. That is, the enzyme-labeled antibody 30s that has not captured the antigen 20s is not moved onto the reactive membrane 8s of the acoustic wave sensor is. Therefore, any reaction is not inhibited by steric hindrance or a substance in a liquid is not captured. As a result, sensing stability and reproducibility are excellent.

A particular example is described below.

The acoustic wave sensor is, as well as a surface acoustic wave device, is manufactured such that the acoustic wave element 2 is formed on the piezoelectric substrate 6 and the insulating film 7s, that is, a SiO₂ film, is then formed. The reactive membrane 8s is formed on the insulating film 7s such that a solution prepared by dissolving a biodegradable plastic in a solvent such as chloroform is applied to the insulating film 7s by spin coating.

An enzyme chemically reacting with the biodegradable plastic is used for the enzyme 31s. A CRP antibody is used for the antibodies 32s and 42s. A CRP antigen is used for the antigen 20s.

A detailed procedure is as described below.

A solution containing magnetic beads, modified with the CRP antibody, having a diameter of about 1 μm is mixed with the CRP antigen (at a concentration of about 1 μg/ml), the CRP antibody labeled with a biodegradable plastic-degrading enzyme, and a blocker solution in a micro-tube. The mixture is agitated for ten minutes, for example, whereby reaction is carried out.

The magnetic beads are collected with a magnet, a supernatant is collected in a pipette, and the remaining magnetic beads are washed with a TBST solution several times.

The magnetic beads washed with the TBST solution are diluted with TBS. An appropriate amount of the dilution is dripped onto the acoustic wave sensor with a pipette. The degradation of a biodegradable plastic membrane by the biodegradable plastic-degrading enzyme is monitored by measuring the change in oscillation frequency of the acoustic wave sensor.

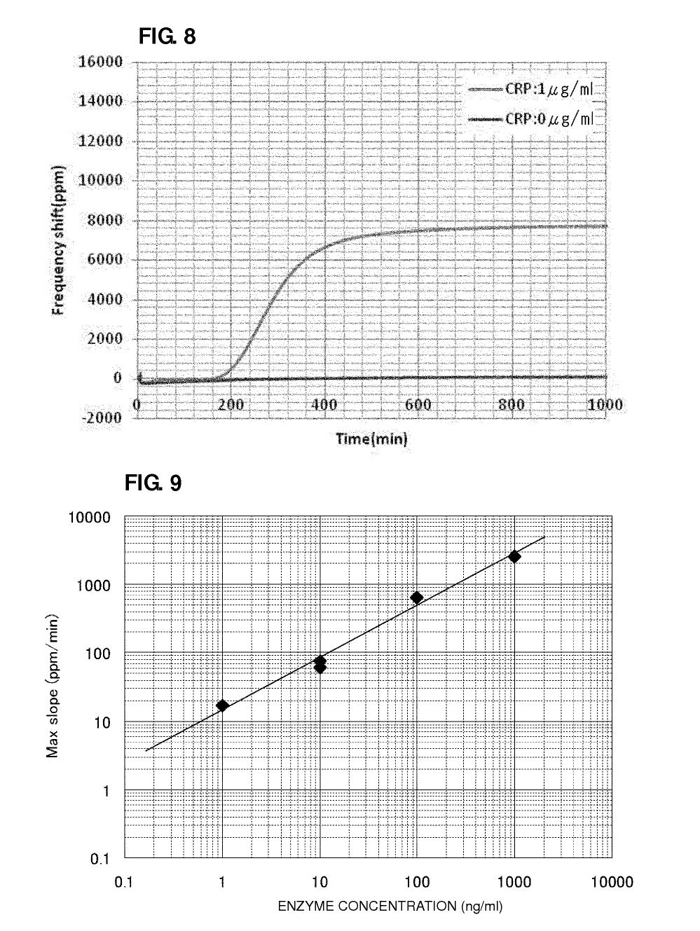

FIG. 8 is a graph showing the change in oscillation frequency thereof. The abscissa represents the elapsed time and the ordinate represents the rate of change in oscillation frequency given by the formula |f₁−f₀|/f₀, where f₀ is the initial frequency and f₁ is the measured frequency. FIG. 8 illustrates the case where the concentration of CRP is 0 μg/ml, that is, the case where the reactive membrane 8s is not removed, and also illustrates the case where the concentration of CRP is 1 μg/ml, that is, the case where the reactive membrane 8s is removed.

FIG. 8 shows that the oscillation frequency varies during the removal of the reactive membrane and becomes constant after the reactive membrane is completely removed.

FIG. 9 is a graph showing the relationship between the maximum rate of change in oscillation frequency and the concentration of an enzyme. The abscissa represents the concentration of the enzyme and the ordinate represents the maximum rate of change in oscillation frequency (that is, the maximum of the rate of change that corresponds to the slope of a change curve as the frequency varies).

FIG. 9 shows that the reaction rate increases with the concentration of the enzyme.

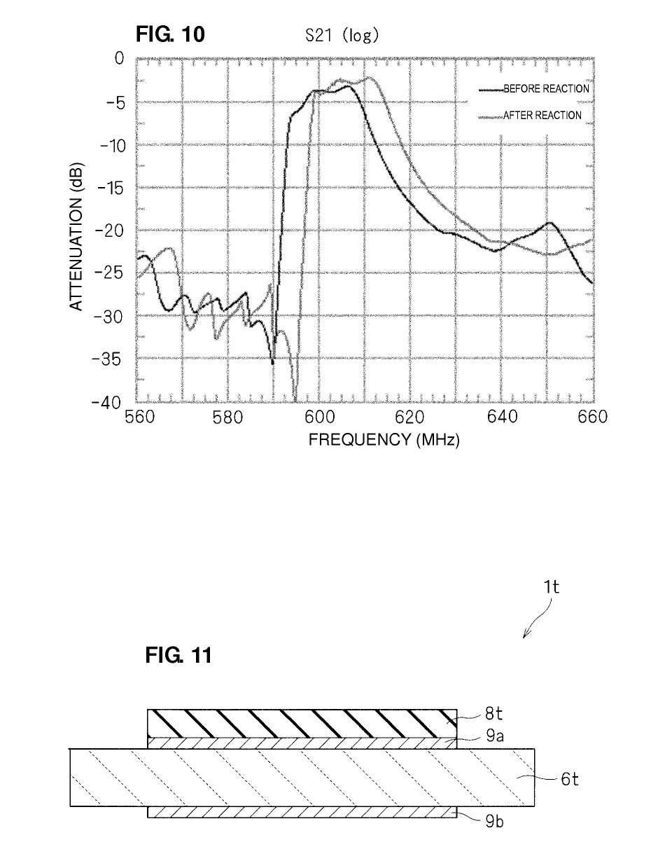

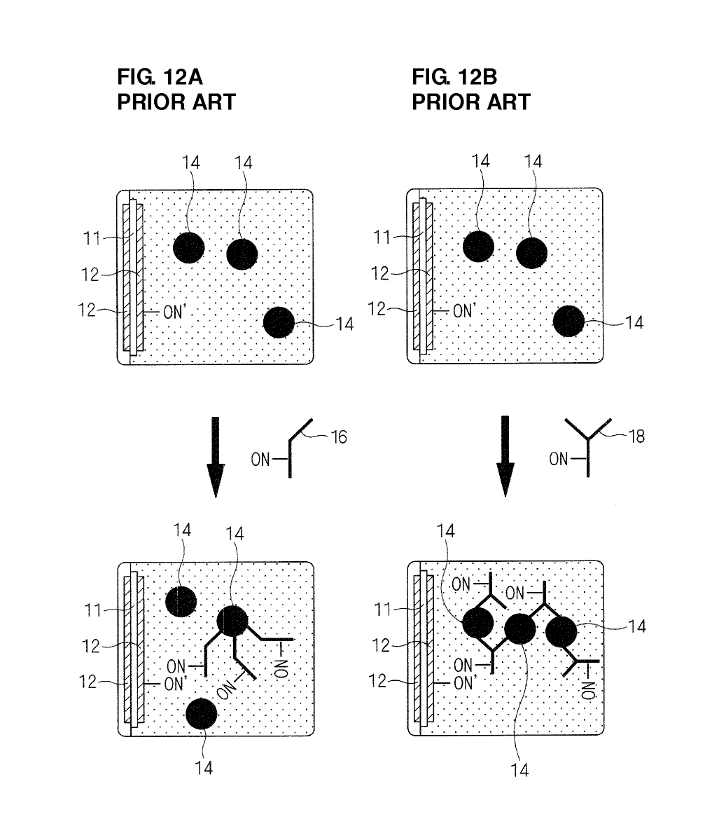

FIG. 10 is a graph showing the frequency response (S21) of the acoustic wave sensor. FIG. 10 illustrates the frequency response of the acoustic wave sensor in which the formed reactive membrane 8s has not reacted, that is, the reactive membrane 8s has not been removed, and also illustrates the frequency response of the acoustic wave sensor in which the formed reactive membrane 8s has been completely removed.

FIG. 10 shows that a change in frequency of about 5 MHz (about 8000 ppm) is achieved without a large loss.

In a comparative example, the change in oscillation frequency of a conventional acoustic wave sensor is observed such that a precipitate produced by the reaction of an enzyme with a substrate is deposited on a sensing surface (a vibration-propagating region in which an acoustic wave propagates) of the conventional acoustic wave sensor. In this case, the precipitate is affected by the propagation condition of the acoustic wave or the like and therefore is not uniformly deposited on the sensing surface and the density of the deposited precipitate is small. This results in that the attenuation of the acoustic wave is large and the frequency range in which oscillation is sustained is narrow.

If a biodegradable plastic is formed into a film by spin coating as described in Example 5, a biodegradable plastic film can be deposited on an acoustic wave resonator so as to have a uniform thickness and a large density. Therefore, the attenuation of an acoustic wave during film formation can be made smaller than that of the comparative example.

The frequency range in which oscillation is sustained depends on the thickness of the biodegradable plastic film. The attenuation of an acoustic wave can be reduced to cause oscillation such that the thickness of the biodegradable plastic film is increased to above the thickness of the precipitate deposited by the method of the comparative example as long as oscillation is sustained. This allows the frequency range in which oscillation is sustained to be wider as compared to the conventional method of depositing the precipitate as described in the comparative example.

### Sixth Preferred Embodiment

An acoustic wave sensor it according to a sixth preferred embodiment of the present invention will now be described with reference to FIG. 11.

FIG. 11 is a sectional view of a principal portion of the acoustic wave sensor it of the sixth preferred embodiment of the present invention. With reference to FIG. 11, the acoustic wave sensor it is a crystal resonator which is configured such that electrodes 9a and 9b are disposed on both surfaces of a crystal substrate 6t and a reactive membrane 8t is disposed on the electrode 9a.

The acoustic wave sensor 1t of the sixth preferred embodiment of the present invention, as well as the acoustic wave sensor 1 of the first preferred embodiment of the present invention, measures the change in oscillation frequency with the change in mass of the reactive membrane 8t by a chemical reaction, whereby the presence or amount of a substance to be detected can be thereby determined.

As described above, an acoustic wave sensor according to various preferred embodiments of the present invention can detect an antigen on the basis of the fact that the reduction in mass of a reactive membrane by the direct or indirect chemical reaction with an enzyme varies the mass applied to an acoustic wave element from the reactive membrane to vary the frequency of the acoustic wave element.

Since the enzyme functions as a catalyst, the chemical reaction of the reactive membrane continues until the reactive membrane is lost. As a result, a larger change in frequency is achieved as compared to the case where an antibody is placed on a conventional acoustic wave element. This allows the acoustic wave sensor to have high sensitivity.

Since the mass of a reactive membrane of an acoustic wave sensor can be reduced by the chemical reaction with an enzyme of an enzyme-labeled antibody that has captured an antigen contained in a sample, the amount of the antigen in the sample can be accurately detected. The type of the antigen in the sample can be accurately determined such that the enzyme-labeled antibody is selected depending on the antigen in the sample. Therefore, sensing stability and reproducibility are excellent.

The present invention is not limited to the above preferred embodiments and various modifications can be made.

For example, a reactive membrane disposed on an acoustic wave element may be one reduced in mass by the direct or indirect chemical reaction with a measured substance or one other than those reduced in mass by the direct or indirect chemical reaction with an enzyme.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. An acoustic wave sensor comprising:
a piezoelectric substrate;
an acoustic wave element including electrodes disposed on the piezoelectric substrate; and
a reactive membrane arranged to over the acoustic wave element and to be reduced in mass by direct or indirect chemical reaction with a measured substance; wherein
the measured substance is detected such that a change in mass applied to the acoustic wave element from the reactive membrane is detected by a change in frequency.

2. The acoustic wave sensor according to claim 1, wherein the measured substance is combined with an enzyme by an immunological method and the mass of the reactive membrane is reduced by the direct or indirect chemical reaction with the enzyme.

3. The acoustic wave sensor according to claim 1, wherein the electrodes are IDT electrodes.

4. The acoustic wave sensor according to claim 1, wherein the acoustic wave element is a surface acoustic wave element.

5. The acoustic wave sensor according to claim 1, wherein the reactive membrane is an organic membrane.

6. The acoustic wave sensor according to claim 5, wherein the reactive membrane is made of a biodegradable plastic.

7. The acoustic wave sensor according to claim 1, wherein the reactive membrane is an inorganic membrane.

8. The acoustic wave sensor according to claim 7, wherein the reactive membrane is a ZnO membrane.
